(19) United States
(12) Patent Application Publication
(10) Pub. No.: US 2016/0120479 A1
(43) Pub. Date: May 5, 2016

(54) Respiration Monitoring Method and Device with Context-Aware Event Classification

(71) Applicant: Sharp Laboratories of America, Inc., Camas, WA (US)

(72) Inventor: Bryan Severt Hallberg, Vancouver, WA (US)

(73) Assignee: Sharp Laboratories of America, Inc., Camas, WA (US)

(21) Appl. No.: 14/529,949

(22) Filed: Oct. 31, 2014

(51) Int. Cl. A61B 5/00; A61B 7/00; A61B 5/08

(52) U.S. Cl. CPC A61B 5/7264; A61B 5/0816; A61B 7/003; A61B 5/7282; A61B 5/7278; A61B 5/7246

(57) ABSTRACT

A respiration monitoring method and device with context-aware noise detection use context supplied by neighboring events when deciding how to classify a target event. More particularly, the present invention considers similarities between a target event and neighboring events relative to event attributes and timing to inform the decision whether to classify the target event as a respiration event or a noise event. Target events classified as noise events are removed from the respiration signal or otherwise ignored when estimating respiration parameters.

### BACKGROUND OF THE INVENTION

The present invention relates to respiration monitoring and, more particularly, improving accuracy in respiration parameter estimation through superior classification of events as between respiration events and noise events.

Respiration is an important vital sign. Abnormal breathing, as reflected by a high or low respiration rate or inspiration to expiration ratio (I:E) or other respiration parameter, can indicate a current or imminent acute health problem, such as an asthma attack or cardiac arrest. Respiration monitoring devices monitor breathing in real-time to enable timely detection of these problems.

Different kinds of respiration monitoring devices are known. One class of devices monitors end-tidal carbon dioxide (EtCO₂) expelled by a patient. Another monitors air pressure through the patient's airways. Another monitors breath sounds emanating from the patient's body. Still others monitor chest movement associated with a patient's breathing using a belt, Doppler detector or video camera. A common feature of respiration monitoring devices is that they generate a signal that is scanned to identify respiration events (i.e. inspiration and expiration) as a function of time from which respiration parameters, such as respiration rate and I:E, can be estimated.

One task performed by respiration monitoring devices that is essential for accurate respiration parameter estimation is proper event classification, that is, distinguishing between respiration events and noise events in the signal. Various techniques have been employed for this purpose. These techniques often scan the signal and classify events having certain threshold attributes, such as a mean amplitude, width and area within a certain range, as respiration events. Events that do not have these threshold attributes are removed from the signal or otherwise ignored when computing the respiration parameters.

A significant limitation of these threshold-driven event classification techniques is their failure to account for contextual differences that can affect how events are expressed in the signal. In some cases, an event having threshold attributes typical of a noise event may be a respiration event. In other cases, an event having threshold attributes typical of a respiration event may be noise emanating from the patient or surrounding environment.

### SUMMARY OF THE INVENTION

The present invention provides a respiration monitoring method and device with context-aware noise detection. The present invention uses context supplied by neighboring events when deciding how to classify a target event. More particularly, the present invention considers similarities between a target event and neighboring events relative to event attributes and timing to inform the decision whether to classify the target event as a respiration event or a noise event. Target events classified as noise events are removed from the respiration signal or otherwise ignored when estimating respiration parameters.

In one aspect of the invention, a respiration monitoring device comprises a body sensor configured to sense respiration activity and generate a respiration signal representing the respiration activity; an event identification engine configured to identify candidate respiration events in the respiration signal; an event classification engine configured to select individual target events from the candidate respiration events, compute an attribute similarity metric for each of the target events by comparing a target value of an attribute of the target event with a reference value for the attribute determined from one or more events neighboring the target event, and classify the target event as one of a noise event or a respiration event using the attribute similarity metric; a respiration parameter estimator configured to compute a respiration parameter estimate using the target events classified as respiration events; and a respiration data output interface configured to output respiration data using the respiration parameter estimate.

In some embodiments, the attribute similarity metric is computed as a quotient of the target value and the reference value.

In some embodiments, the target event is classified using a result of a comparison of the attribute similarity metric with a threshold value.

In some embodiments, the target event is classified using a result of a comparison of the attribute similarity metric and a timing similarity metric with a threshold value.

In some embodiments, the reference value is selected as a value of the attribute for one of the neighboring events which is most similar to the values of the attribute for the other neighboring events.

In some embodiments, the attribute is mean event amplitude.

In some embodiments, the attribute is event width.

In some embodiments, the attribute is event area.

In some embodiments, the timing similarity metric is computed using a respiration period and respiration phase interval associated with the target event.

In some embodiments, the body sensor is a sound sensor.

In some embodiments, the respiration parameter is respiration rate.

In some embodiments, the event classification engine is further configured to be able to classify at least one of the candidate respiration events as a noise event using a result of a comparison of a value of an attribute of the candidate respiration event with a threshold value of the attribute.

In another aspect of the invention, respiration monitoring method for a respiration monitoring device having a body sensor comprises sensing, by the body sensor, respiration activity; generating, by the device, a respiration signal representing the respiration activity; identifying, by the device, candidate respiration events in the respiration signal; selecting, by the device, individual target events from the candidate respiration events; computing, by the device, an attribute similarity metric for each of the target events by comparing a target value of an attribute of the target event with a reference value for the attribute determined from one or more events neighboring the target event; classifying, by the device, the target event as one of a noise event or a respiration event using the attribute similarity metric; computing, by the device, a respiration parameter estimate using the target events classified as respiration events; and outputting, by the device, respiration data using the respiration parameter estimate.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a respiration monitoring device.

FIG. 2 shows an event classification engine.

FIG. 3 shows threshold noise event detection checks performed by an event classification engine.

FIG. 4 shows contextual noise event detection checks performed by an event classification engine.

FIG. 5 shows a generic contextual noise event detection check performed by an event classification engine.

FIG. 6 shows a generic method for determining a reference value of an attribute from neighboring events.

FIG. 7A shows a method for computing a period similarity metric for a target event.

FIG. 7B shows a method for computing an interval similarity metric for a target event.

FIG. 8 shows designation of periods between neighboring events in a buffered segment when an event is ignored.

FIG. 9 shows designation of periods between neighboring events in a buffered segment when the event is counted.

FIG. 10 shows designation of intervals between neighboring events in a buffered segment when an event is ignored.

FIG. 11 shows designation of intervals between neighboring events in a buffered segment when the event is counted.

FIG. 12 shows nearby events subject to contextual nearby small event detection.

### DETAILED DESCRIPTION OF A PREFERRED EMBODIMENT

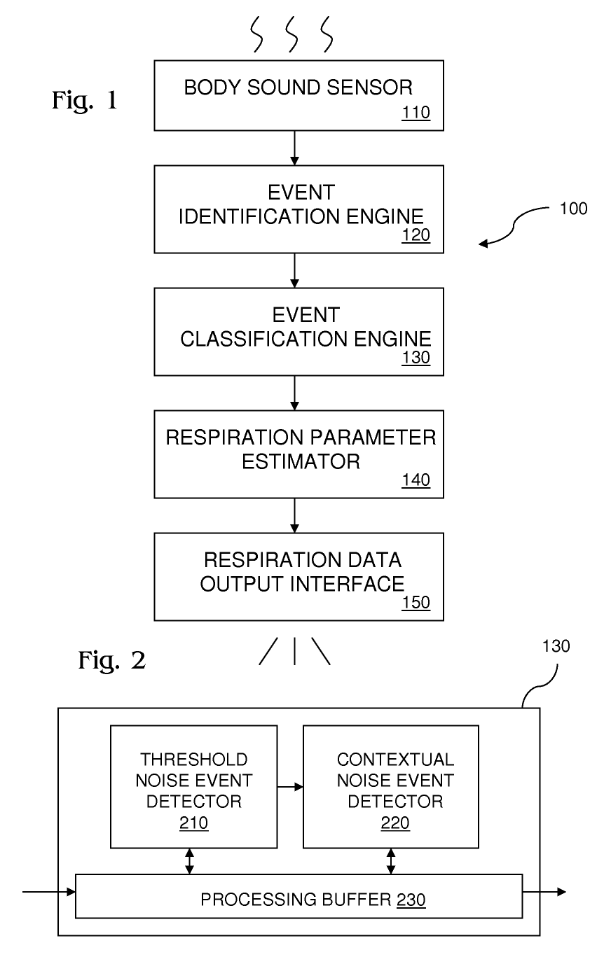

FIG. 1 shows a respiration monitoring device 100 in some embodiments of the invention. Device 100 includes a body sound sensor 110, an event identification engine 120, an event classification engine 130, a respiration parameter estimator 140 and a respiration data output interface 150, which are serially communicatively coupled.

In operation, sensor 110 is positioned on or adjacent the body of a human subject, such as at the subject's chest or tracheal notch. Sensor 110 has a sound transducer, such as a microphone, that continually detects local sound waves including breath sounds as well as noise, converts the sound waves into digital samples representing the sounds and generates a continuous respiration signal from the digital samples. In addition to a sound transducer, sensor 110 may include one or more amplifiers, filters and an analog/digital converter which assist in generating the respiration signal. Sensor 110 transmits the respiration signal to identification engine 120. The respiration signal received by identification engine 120 expresses the subject's breathing sequence in the form of inspiration and expiration events as well as expressing randomly occurring noise events, such as coughing, talking and other impulse sounds.

Identification engine 120 filters and rectifies the respiration signal and generates an energy envelope of the signal. Identification engine 120 may perform other preparatory operations on the respiration signal, such as noise spike removal. Identification engine 120 proceeds to identify candidate respiration events in the respiration signal by scanning the signal for attributes and patterns that match respiration event definitions. Candidate respiration events include candidate inspiration events and candidate expiration events. In some embodiments, identification engine 120 identifies candidate respiration events in the respiration signal as described in application Ser. No. 14/231,275 entitled “METHOD AND ENGINE FOR DEFINING RESPIRATION EVENTS IN BODY SENSOR SIGNALS,” filed Mar. 31, 2014 (hereinafter “the '275 Application”), which is commonly owned with the present application and is incorporated herein by reference. Identification engine 120 conveys results of candidate respiration event identification to classification engine 130. For example, identification engine 120 may store information identifying the boundaries of candidate respiration events in a candidate event list that is accessible to classification engine 130.

FIG. 2 shows classification engine 130 in more detail to include a threshold noise event detector 210 and a contextual noise event detector 220 which operate sequentially on a segment of the respiration signal presently stored in a processing buffer 230. Buffer 230 is a storage element that holds a predetermined number of seconds of the respiration signal energy envelope, such as 30 seconds. Buffer 230 may be a rolling buffer where every predetermined number of seconds, such as every one second, the oldest portion of the buffered segment is replaced with a new portion. The buffered segment includes a number of candidate respiration events identified by identification engine 120. In some embodiments of the invention, candidate respiration events are considered neighboring events by virtue of being part of the same buffered segment (e.g., part of the same 30-second window).

Threshold detector 210 detects obvious noise events among the candidate respiration events within the buffered segment by identifying threshold violations. Detector 210 detects events having attributes that are physiologically impossible or highly improbable and can therefore be classified as noise without the need to consider context supplied by neighboring events. Detector 210 compares values of different attributes of candidate respiration events with maximum and minimum threshold values for those attributes. The threshold values are set to levels that are physiologically impossible or highly improbable for respiration events. When a value of an attribute of a candidate respiration event is above the maximum threshold or below the minimum threshold value for the attribute, the event is labeled as a noise event and is either removed from the respiration signal or otherwise ignored by subsequent processing elements, such as context detector 220 and respiration parameter estimator 140.

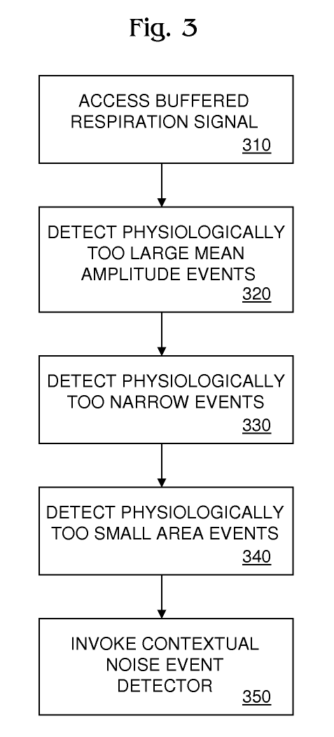

FIG. 3 shows threshold noise event detection checks performed by threshold detector 210. Detector 210 accesses the buffered segment (310) and detects among the candidate respiration events identified by identification engine 120 events having mean amplitudes that are physiologically too large to be respiration events (320). Physiologically too large mean amplitude events are events, such as coughing or talking, that are too loud to be respiration events regardless of context. In this check, the mean amplitude of an event Mean_{Target} is compared with a predetermined multiple of a local noise threshold Mean_{NoiseFloor} near the event. In some embodiments, the local noise threshold is computed as described in the '275 Application. If the mean amplitude is greater than the predetermined multiple of the local noise threshold, the event is labeled a noise event and is removed from the signal or is otherwise ignored by subsequent processing elements. The predetermined multiple may be set to 300, in which case the check is expressed mathematically as

Delete=(Mean_{Target}>(300*Mean_{Noise Floor}))

Next, detector 210 detects among the remaining candidate respiration events in the buffered segment events that are physiologically too narrow to be respiration events (330). Physiologically too narrow events are events that have too short a duration to be respiration events, based on the empirical observation that breathing takes a minimum amount of time to perform. In this check, the width of an event Width_{Target}, which represents the duration of the event, is compared with a predetermined width threshold. If the event width is less than the predetermined width threshold, the event is labeled a noise event and is removed from the signal or otherwise ignored by subsequent processing elements. The predetermined multiple may be set to 100 milliseconds, in which case the check is expressed mathematically as

Delete=(Width_{Target}<100 mS)

Next, detector 210 detects among the remaining candidate respiration events in the buffered segment events having physiologically too small of an area to be respiration events (330). Physiologically too small area events are events that have too small of an area to be respiration events, based on the empirical observation that breathing requires a minimum amount of air to be transported through the trachea, which is roughly equal to the area represented by the product of the mean amplitude and the duration of the event. In this check, the product of the mean amplitude of an event Mean_{Target} and the width of the event Width_{Target} is compared with a predetermined multiple of a local noise threshold Mean_{NoiseFloor} near the event. If the product is greater than the predetermined multiple of the local noise threshold, the event is labeled a noise event and is removed from the signal or otherwise ignored by subsequent processes. The predetermined multiple may be set to 200 milliseconds, in which case the check is expressed mathematically as

Delete=((Mean_{Target}*Width_{Target})>(Mean_{Noise Floor}*200 mS))

In some embodiments, the threshold checks described above are performed in a different order.

Contextual detector 220 detects further noise events among the remaining candidate respiration events within the buffered segment using context supplied by neighboring events. In a respiration signal, respiration events which are close in time tend to have similar attributes and timing patterns. Context detector 220 considers similarities in terms of attributes and timing patterns between target events being evaluated and neighboring events to detect events that are likely to be noise given the extent of dissimilarity with their neighbors. When a candidate respiration event is labeled as noise event through contextual noise event detection, the event is removed from the respiration signal or otherwise ignored by subsequent processing elements including respiration parameter estimator 140.

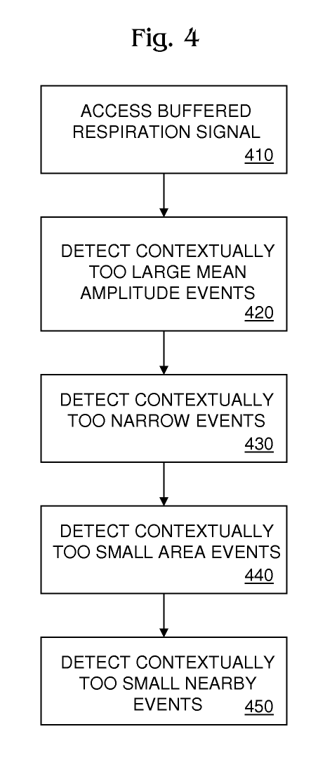

FIG. 4 shows contextual noise event detection checks performed by contextual detector 220. Detector 220 accesses the buffered segment (410) and detects events among the candidate respiration events remaining after threshold noise event detection that are too large to be respiration events in their present context (420). Next, detector 220 detects contextually too narrow events among the remaining candidate respiration events (430). Next, detector 220 detects contextually too small area events among the remaining candidate respiration events (440). Finally, detector 220 detects contextually too small nearby events among the remaining candidate respiration events (450). The events detected through each contextual check are labeled as noise events and are removed from the signal or are otherwise ignored by subsequent processing elements.

Contextual detector 220 performs contextual noise event detection checks on each candidate respiration event within the buffered segment that was not labeled a noise segment by threshold detector 210. Moreover, each check is performed twice on each event, both using past context from neighboring events that precede the event and using future context from neighboring events that follow the event. Detector 220 labels an event as a noise event only if the event fails both instances of the check.

While the different contextual noise event detection checks performed by detector 220 evaluate different attributes, such as mean amplitude, width and area, a common objective of these checks is determining the similarity between a target event being evaluated and neighboring events relative to the attribute under consideration. As a result, these checks have certain generic features that can be abstracted, such as calculating a reference value for the attribute from neighboring events, calculating an attribute similarity metric between the target event and neighboring events relative to the attribute using the reference value and labeling the target event as a noise event if the target event and the neighboring events are too dissimilar with regard to the attribute.

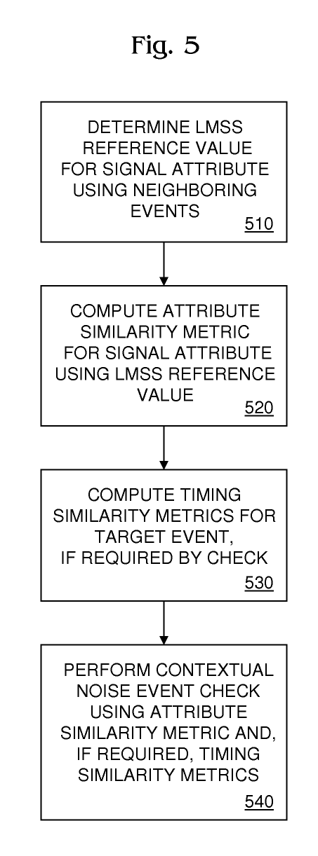

FIG. 5 shows a generic contextual noise event detection check performed by detector 220. First, detector 220 determines a largest most similar segment (LMSS) reference value for the attribute under consideration (e.g., mean amplitude, width, area) from events neighboring the target event (510). To determine the LMSS reference value, each neighboring event is individually selected as an LMSS candidate and a group similarity metric is calculated for the LMSS candidate by comparing the LMSS candidate individually with the other neighboring events for similarity with regard to the attribute and combining the results. More particularly, the group similarity metric for an LMSS candidate is computed as the normalized, weighted sum of the individual similarity metrics between the LMSS candidate and the other neighboring events. The LMSS candidate having the highest group similarity metric is selected as the LMSS and its value relative to the attribute is selected as the LMSS reference value.

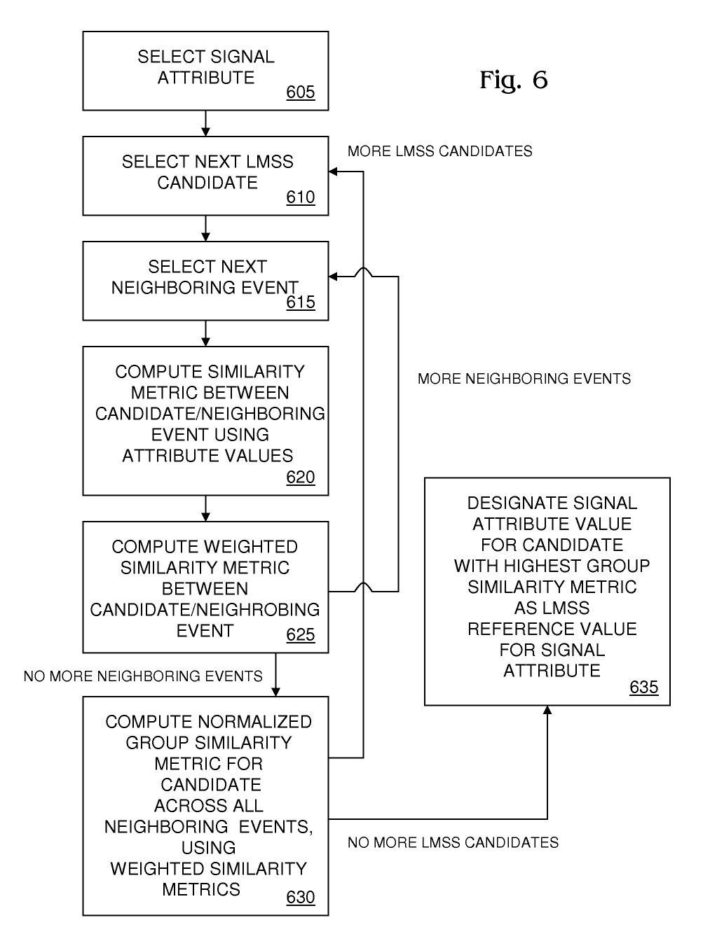

Turning to FIG. 6, a generic method for determining an LMSS reference value is shown. The attribute under consideration is selected (605) and a neighboring event to the target event is selected as the first LMSS candidate (610). A second neighboring event is then selected for comparison with the LMSS candidate (615) and the value of the attribute for the LMSS candidate is compared for similarity with the value of the attribute for the neighboring event, resulting in a similarity metric between the LMSS candidate and the neighboring event with regard to the attribute being computed (620). The similarity metric Similarity(x,y) is computed by dividing the smaller of the values of the attribute min(x,y) as between the LMSS candidate and the neighboring event by the larger of the values of the attribute max(x,y) as between the LMSS candidate and the neighboring event. Accordingly, the value of an unweighted similarity metric is always between zero and one, inclusive, and can be expressed mathematically as

$$Similarity ( x , y ) = \frac{min ( x , y )}{max ( x , y )}$$

The similarity metric is then weighted by multiplying the similarity metric by the mean amplitude of the neighboring event (625), which favors higher amplitude events that are less likely to be noise in the LMSS selection process. Steps 615-625 are then repeated to compare the LMSS candidate with other neighboring events. Once weighted similarity metrics have been computed between the LMSS candidate and all other neighboring events, a normalized group similarity metric is computed for the LMSS candidate across all of the neighboring events as the normalized sum of all weighted similarity metrics (630). Steps 610-630 are then repeated considering other neighboring events as LMSS candidates. Once group similarity metrics have been computed for all LMSS candidates, the LMSS candidate having the highest group similarity metric is selected as the LMSS and its value in relation to the attribute is selected as the LMSS reference value (635).

Returning to FIG. 5, detector 220 next computes an attribute similarity metric for the target event using the LMSS reference value (520). The attribute similarity metric is computed by dividing the smaller value of the attribute as between the LMSS candidate and the target event by the larger value of the attribute as between the LMSS candidate and the target event. Accordingly, the value of the attribute similarity metric is always between zero and one, inclusive. As discussed, the attribute evaluated for similarity may be, for example, mean amplitude, width or area.

Next, detector 220 computes a timing similarity metric for the target event, if required by the particular contextual noise event check being performed (530). In a respiration signal, respiration events which are close in time tend to not only have similar attributes but also tend to exhibit regular timing patterns, such as regular respiration periods and respiration phase intervals. Accordingly, certain contextual noise event detection checks consider a timing similarity metric in conjunction with an attribute similarity metric to inform the decision whether to classify the target event as a noise event. The timing similarity metric indicates whether the target event makes timing patterns more or less regular, which informs whether the target event is a respiration event or a noise event.

Because the timing similarity metric reflects how the target event affects both the respiration period and the respiration phase interval, period and interval similarity metrics are first separately computed and are then combined to generate the timing similarity metric. Moreover, the timing similarity metric is computed twice for each target event, both using context from neighboring events that precede the target event to compute a pre-event timing similarity metric and using context from neighboring events that follow the target event to compute a post-event timing similarity metric.

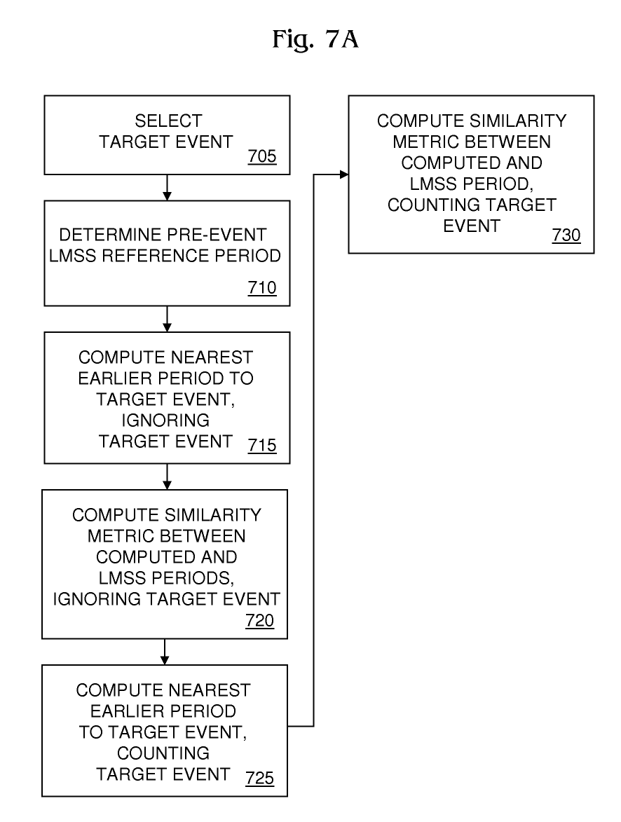

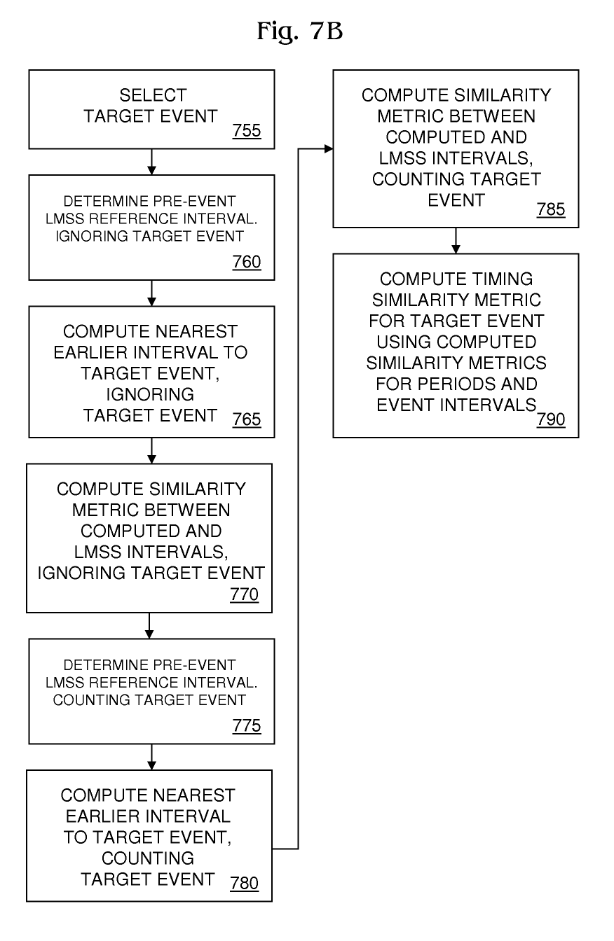

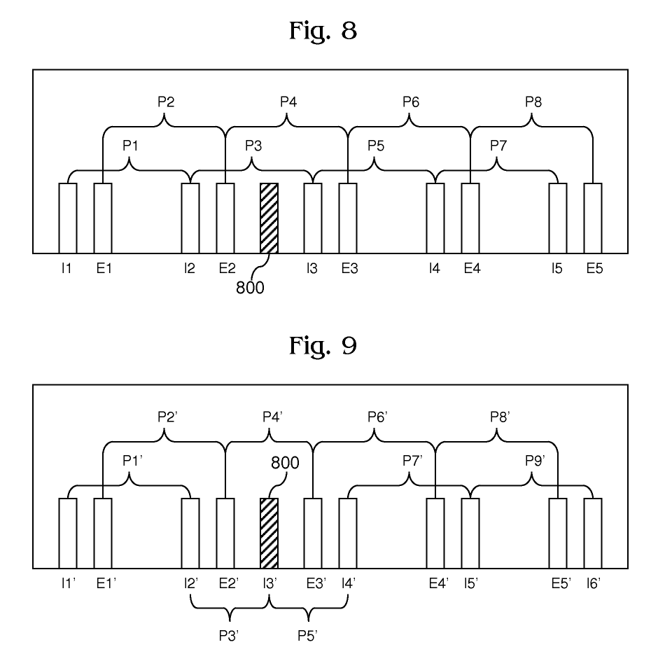

Turning now to FIG. 7A, a generic method for computing a period similarity metric for a target event is shown. This method will be described in conjunction with a buffered segment shown in FIGS. 8 and 9. The method describes computation of the pre-event period similarity metric for the target event, although analogous steps are performed to compute a post-event period similarity metric for the target event.

At the outset of the method, detector 220 selects the target event being evaluated (705) and determines the pre-event LMSS reference period (710). The pre-event LMSS reference period is determined by computing a group similarity metric for each pre-event period individually and designating the period having the highest group similarity metric as the LMSS reference period. For example, in FIG. 8, the periods that are pre-event LMSS candidates include a first period P1 between inspiration event I1 and inspiration event I2 and a second period P2 between expiration event E1 and expiration event E2, both of which precede a target event 800 that is initially presumed to be a noise event. Since the group similarity metrics for P1 and P2 are substantially identical, the LMSS reference period is essentially the same regardless of whether P1 or P2 is selected as the LMSS and has a value roughly equal to both P1 and P2.

Next, detector 220 computes the nearest earlier period to the target event, ignoring the target event as noise (715). For example, returning to FIG. 8, the earlier period nearest target event 800 that disregards target event 800 is P3, which begins at inspiration event I2 and ends at inspiration event I3.

Next, detector 220 computes the similarity metric between the period computed in Step 715 and the LMSS reference period computed in Step 710 (720). In particular, detector 220 computes a similarity metric deleted_period_similarity by dividing P3 by the LMSS reference period (P1 or P2). This quotient has a value close to one, indicating that the respiration period of the buffered segment is regular if target event 800 is ignored as a noise event.

Next, detector 220 computes the nearest earlier period to the target event, this time counting the target event as a valid respiration event that must be accounted for (725). For example, in FIG. 9, the earlier period nearest target event 800 that counts target event 800 is P3′, which begins at inspiration event I2′ and ends at inspiration event I3′.

Next, detector 220 computes the similarity metric between the period computed in Step 725 and the LMSS reference period computed in Step 710 (730). In particular, detector 220 computes a similarity metric kept_period_similarity by dividing P3′ by the LMSS reference period (P1′ or P2′). This quotient has a value close to 0.67, indicating that the respiration period of the buffered segment is irregular if target event 800 is counted as a respiration event.

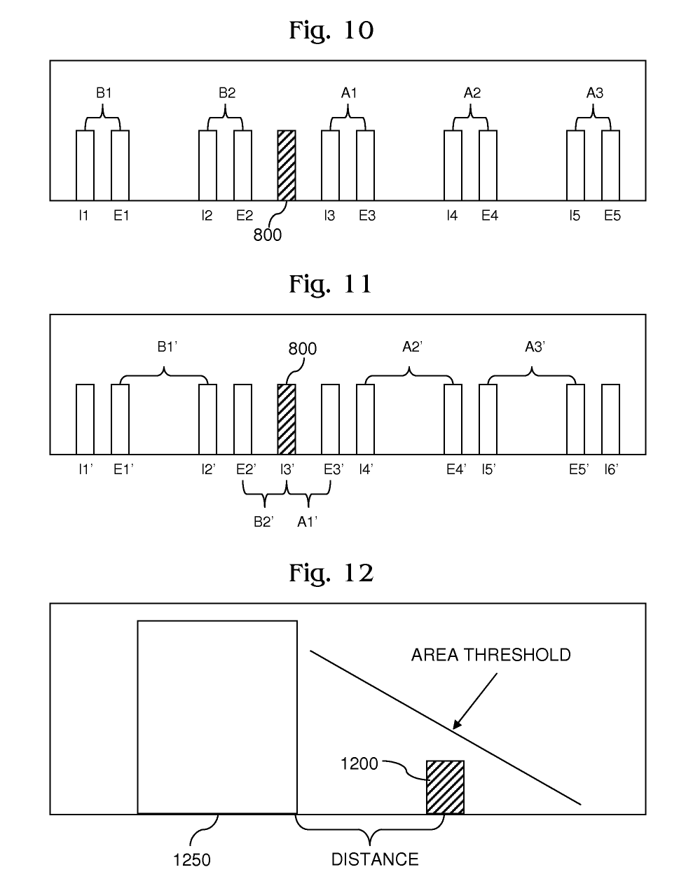

Turning now to FIG. 7B, a generic method for computing an interval similarity metric for a target event is shown. This method will be described in conjunction with a buffered segment shown in FIGS. 10 and 11. The method describes computation of the pre-event interval similarity metric for the target event, although analogous steps are performed to compute the post-event interval similarity metric for the target event.

At the outset of the method, detector 220 selects the target event being evaluated (755) and determines the pre-event LMSS reference interval, ignoring the target event as noise (760). This pre-event LMSS reference interval is determined by computing a group similarity metric for each pre-event interval individually and designating the interval having the highest group similarity metric as the LMSS reference interval. For example, in FIG. 10, the intervals that are pre-event LMSS candidates include a first interval B1 between inspiration event I1 and expiration event E1 and a second interval B2 between inspiration event I2 and expiration event E2, both of which precede target event 800 that is initially assumed to be a noise event. Since the group similarity metrics for B1 and B2 are substantially identical, the LMSS reference interval is essentially the same regardless of whether B1 or B2 is selected as the LMSS and has a value roughly equal to both B1 and B2.

Next, detector 220 computes the nearest earlier interval to the target event, again ignoring the target event as noise (765). For example, returning to FIG. 10, the earlier interval nearest target event 800 that disregards target event 800 is B2, which begins at inspiration event I2 and ends at expiration event E2.

Next, detector 220 computes the pre-event similarity metric between the interval computed in Step 765 and the LMSS reference interval computed in Step 760 (770). In particular, detector 220 computes a similarity metric deleted_adjacent_similarity by dividing B2 by the LMSS reference interval (B1 or B2). This quotient has a value close to one, confirming that the respiration interval of the buffered segment is regular if target event 800 is ignored as a noise event.

Next, detector 220 determines the pre-event LMSS reference interval, this time counting the target event as a respiration event (775). This pre-event LMSS reference interval is determined by computing a group similarity metric for each pre-event interval individually and designating the interval having the highest group similarity metric as the LMSS reference interval. In FIG. 11, the sole pre-event interval is interval B1′ between expiration event E1′ and inspiration event I2′. Accordingly, the LMSS reference interval has a value equal to B1′.

Next, detector 220 computes the nearest earlier interval to the target event, again counting the target event as a respiration event (780). For example, returning to FIG. 11, the earlier interval nearest target event 800 when target event 800 is considered is B2′, which begins at expiration event E2′ and ends at inspiration event I3′.

Next, detector 220 computes the pre-event similarity metric between the interval computed in Step 780 and the LMSS reference interval computed in Step 775 (785). In particular, detector 220 computes a similarity metric kept_adjacent_similarity by dividing B2′ by the LMSS reference interval B1′. This quotient has a value close to 0.5, confirming that the respiration interval of the buffered segment is irregular if target event 800 is counted as a respiration event.

Finally, detector 220 computes the pre-event timing similarity metric for the target event by combining the four pre-event similarity metrics computed as described in FIGS. 7A and 7B (790). More particularly, detector 220 computes a timing similarity metric Timing Similarity according to the following formula:

$$Timing Similarity = ( \frac{{kept}_{{period}_{similarity}} * {kept}_{{adjacent}_{similiarity}}}{{deleted}_{{period}_{similarity}} * {deleted}_{{adjacent}_{similarity}}} )$$

It bears noting that the above equation yields a value greater than one when counting the target event as a respiration event produces a more regular timing pattern, and yields a value less than one when disregarding the target event as noise produces a more regular timing pattern. In the case of target event 800 shown in FIGS. 8-11, the pre-event timing similarity is approximately 0.34, indicating that ignoring target event 800 as noise produces a more regular timing pattern.

The post-event timing similarity metric for the target event is computed using post-event periods and intervals (e.g., P5-P9, A1-A3, etc.) in a manner analogous to how the pre-event timing similarity metric is computed.

Returning to FIG. 5, detector 220 completes the generic contextual noise event detection check using the attribute similarity metric computed in Step 520 and, where the check requires consideration of timing similarity, the timing similarity metric computed in Step 530. As mentioned, detector 220 performs a given check twice on the target event, both using past context from neighboring events that precede the target event and using future context from neighboring events that follow the target event, and labels the target event as a noise event only if the target event fails both the past and future instances of the check.

With the discussion of the generic check complete, the specific contextual noise event detection checks identified in FIG. 4 will now be explained in greater detail.

The contextually too large mean amplitude event check of Step 420 recognizes that target events which have (1) a larger mean amplitude Mean_{Target} than the LMSS reference mean amplitude Mean_{LMSS}, (2) a mean amplitude attribute similarity metric Similarity(Mean_{Target},Mean_{LMSS}) less than a predetermined threshold value, preferably 0.1, and (3) a mean amplitude Mean_{Target} larger than a predetermined threshold multiple, preferably 200, of the local noise floor Mean_{Target} are very large compared with neighboring respiration events and thus should be classified as noise events and ignored by subsequent processing elements. This check is expressed in the following mathematical formulation:

$$Delete = ( \frac{{Mean}_{Target}}{{Mean}_{LMSS}} > 1 ) \&\& ( Similari ty ( {Mean}_{Target} , {Mean}_{LMSS} ) < 0.1 ) \&\& ( \frac{{Mean}_{Target}}{{Mean}_{Noise Floor}} > 200 )$$

The contextually too narrow event check of Step 430 recognizes that target events which have (1) a narrower width Width_{Target} than the LMSS reference width Width_{LMSS}, (2) a width attribute similarity metric Similarity(Width_{Target}, Width_{LMSS}) less than a predetermined threshold value, preferably 0.3, and (3) a timing similarity metric Timing Similarity and width attribute similarity metric Similarity(Width_{Target}, Width_{LMSS}) product less than a predetermined threshold value, preferably 0.3, are very narrow and poorly timed compared with neighboring respiration events and thus should be classified as noise events and ignored by subsequent processing elements. This check is expressed in the following mathematical formulation:

$$Delete = ( \frac{{Width}_{Target}}{{Width}_{LMSS}} < 1 ) \&\& ( Similari ty ( {Width}_{Target} , {Width}_{LMSS} ) < 0.3 ) \&\& ( TimingSimilarity * Similarity ( {Width}_{Target} , {Width}_{LMSS} ) < 0.3 )$$

The contextually too small area event check of Step 440 recognizes that target events which have (1) a smaller area Area_{Target} than the LMSS reference area Area_{LMSS}, (2) an area attribute similarity metric Similarity(Area_{Target}, Area_{LMSS}) less than a predetermined threshold value, preferably 0.1, and (3) a timing similarity metric Timing Similarity and area attribute similarity metric Similarity(Area_{Target}, Area_{LMSS}) product less than a predetermined threshold value, preferably 0.1, are very small and poorly timed compared with neighboring respiration events and thus should be classified as noise events and ignored by subsequent processing elements. This check is expressed in the following mathematical formulation:

$$Delete = ( \frac{{Area}_{Target}}{{Area}_{LMSS}} < 1 ) \&\& ( Similari ty ( {Area}_{Target} , {Area}_{LMSS} ) < 0.1 ) \&\& ( TimingSimilarity * Similarity ( {Area}_{Target} , {Area}_{LMSS} ) < 0.1 )$$

Finally, the contextually too small nearby event check of Step 450 recognizes that smaller events which occur too near larger respiration events cannot themselves be respiration events and should be classified as noise events and ignored by subsequent processing elements. These too small nearby events are detected using an area threshold AreaThreshold that is large near the larger event and diminishes over time. The area threshold at the boundary of the larger event is the product of the larger event's mean amplitude Mean_{LargerEvent} and width Width_{LargerEvent} (or Width_{Larger}) divided by a scaling factor, preferably 20. The area threshold diminishes at a rate equal to the distance between the larger event and the smaller event Distance_{LargerToSmaller}, divided by the product of the larger event's width and a scaling factor, preferably 3.0. The area threshold computation is expressed in the following formula:

$$Area Threshold = ( \frac{{Mean}_{LargerEvent} * {Width}_{LargerEvent}}{20} ) * ( 1 - \frac{{Distance}_{LargerToSmaller}}{3 * {Width}_{Larger}} )$$

The smaller event is classified as a noise event if the smaller event has an area Area_{Smaller} smaller than the area threshold and a timing similarity metric Timing Similarity less than 1.0. The check is expressed in the following formula:

Delete=(Area_{Smaller}<AreaThreshold)&&(TimingSimilarity<1)

The small nearby event case is illustrated in FIG. 12, by way of example, where a smaller event 1200 is separated from a larger respiration event 1250 by a short distance and is below an area threshold that is large near the larger event 1250 and diminishes over time.

Returning now to FIG. 1, classification engine 130 conveys the results of event classification to respiration parameter estimator 140. For example, classification engine 130 may remove events classified as noise events from the buffered segment and provide estimator 140 access to the buffered segment. Alternatively, classification engine 130 may store information identifying events classified as noise events in a noise event list that is made accessible to estimator 140 along with the buffered segment. In another alternative, classification engine 130 may store information identifying events not classified as noise events in a respiration event list that is made accessible to estimator 140 along with the buffered segment. Regardless of the manner in which respiration parameter estimator 140 is made aware of the results of event classification, estimator 140 applies the results to generate respiration parameter estimates, such as respiration rate and I:E estimates, and transmits to respiration data output interface 150 respiration data generated using the estimates, which may include the estimates themselves and respiration data derived from the estimates. Data output interface 150 may display the respiration data locally, relay them to a remote clinician facility, or both.

Identification engine 120, classification engine 130 and estimator 140 may perform their respective tasks through the execution of software instructions on a general purpose processor, through the execution of instructions by a microcontroller, in custom circuitry, or using a combination of one or more of the above.

It will be appreciated by those of ordinary skill in the art that the invention can be embodied in other specific forms without departing from the spirit or essential character hereof. For example, the respiration signal can be acquired using a non-acoustic body sensor, such as a sensor that measures end-tidal carbon dioxide (EtCO₂) expelled by the subject, air pressure through the subject's airways or chest movement associated with the subject's breathing using a belt, Doppler detector or video camera. The threshold values used in the checks may require adjustment where these alternative body sensors are used. Moreover, the invention may be applied to improve parameter extraction from other types of contextually stable signals that include randomly occurring noise, such as electrocardiogram signals and walking sensor signals. The present description is therefore considered in all respects to be illustrative and not restrictive. The scope of the invention is indicated by the appended claims, and all changes that come within the meaning and range of equivalents thereof are intended to be embraced therein.

What is claimed is:

1. A respiration monitoring device, comprising:
a body sensor configured to sense respiration activity and generate a respiration signal representing the respiration activity;
an event identification engine configured to identify candidate respiration events in the respiration signal;
an event classification engine configured to select individual target events from the candidate respiration events, compute an attribute similarity metric for each of the target events by comparing a target value of an attribute of the target event with a reference value for the attribute determined from one or more events neighboring the target event, and classify the target event as one of a noise event or a respiration event using the attribute similarity metric;
a respiration parameter estimator configured to compute a respiration parameter estimate using the target events classified as respiration events; and
a respiration data output interface configured to output respiration data using the respiration parameter estimate.

2. The device of claim 1, wherein the attribute similarity metric is computed as a quotient of the target value and the reference value.

3. The device of claim 1, wherein the target event is classified using a result of a comparison of the attribute similarity metric with a threshold value.

4. The device of claim 1, wherein the target event is classified using a result of a comparison of the attribute similarity metric and a timing similarity metric with a threshold value.

5. The device of claim 1, wherein the reference value is selected as a value of the attribute for one of the neighboring events which is most similar to the values of the attribute for the other neighboring events.

6. The device of claim 1, wherein the attribute is mean event amplitude.

7. The device of claim 1, wherein the attribute is event width.

8. The device of claim 1, wherein the attribute is event area.

9. The device of claim 1, wherein the timing similarity metric is computed using a period and phase interval associated with the target event.

10. The device of claim 1, wherein the body sensor is a sound sensor.

11. The device of claim 1, wherein the respiration parameter is respiration rate.

12. The device of claim 1, wherein the event classification engine is further configured to be able to classify at least one of the candidate respiration events as a noise event using a result of a comparison of a value of an attribute of the candidate respiration event with a threshold value of the attribute.

13. A respiration monitoring method for a respiration monitoring device having a body sensor, comprising:
sensing, by the body sensor, respiration activity;
generating, by the device, a respiration signal representing the respiration activity;
identifying, by the device, candidate respiration events in the respiration signal;
selecting, by the device, individual target events from the candidate respiration events;
computing, by the device, an attribute similarity metric for each of the target events by comparing a target value of an attribute of the target event with a reference value of the attribute determined from one or more events neighboring the target event;
classifying, by the device, the target event as one of a noise event or a respiration event using the attribute similarity metric;
computing, by the device, a respiration parameter estimate using the target events classified as respiration events; and
outputting, by the device, respiration data using the respiration parameter estimate.

14. The method of claim 13, wherein the attribute similarity metric is computed as a quotient of the target value and the reference value.

15. The method of claim 13, wherein the target event is classified using a result of a comparison of the attribute similarity metric with a threshold value.

16. The method of claim 13, wherein the target event is classified using a result of a comparison of the attribute similarity metric and a timing similarity metric with a threshold value.

17. The method of claim 13, wherein the reference value is selected as a value of the attribute for one of the neighboring events which is most similar to the values of the attribute for the other neighboring events.

18. The method of claim 13, wherein the attribute is one of mean event amplitude, event width or event area.

19. The method of claim 13, wherein the timing similarity metric is computed using a period and phase interval associated with the target event.

20. The method of claim 13, wherein the body sensor is a sound sensor.
